(19) United States
(12) Patent Application Publication
(10) Pub. No.: US 2024/0138687 A1
(43) Pub. Date: May 2, 2024

(54) Cuffless Blood Pressure Measurement Apparatus and Method

(71) Applicants: Nikita Balashov, Salt Lake City, UT (US); Vadym Danylevych, Chortkiv (UA)

(72) Inventors: Nikita Balashov, Salt Lake City, UT (US); Vadym Danylevych, Chortkiv (UA)

(21) Appl. No.: 18/050,493

(22) Filed: Oct. 28, 2022

(51) Int. Cl. A61B 5/021; A61B 5/00; A61B 7/04

(52) U.S. Cl. CPC A61B 5/02141; A61B 5/0004; A61B 5/6843; A61B 5/7257; A61B 5/7267; A61B 7/045; A61B 2562/0247

(57) ABSTRACT

Cuffless apparatus comprised of a pressure applicator operated at the wrist or other pulse-taking location and a processing unit implementing a method of measurement of arterial blood pressure and detection of auscultatory gap. Applanation pressure is applied by user's fingers in a pulsatile fashion as per real-time instructions of the processing unit. Pressure magnitude and sound field parameters captured by sensors are used to estimate systolic and diastolic blood pressure by analysis of Korotkoff sounds. User instructions are adjusted during measurement to facilitate self-validation by correlating results of two machine learning approaches: recognition of correlation of derivatives of the pressure and sound amplitude as functions of time, and image recognition of Korotkoff events in sound spectrograms overlayed with pressure traces. The auscultatory gap is detected when sound intensity is reduced by at least forty percent for one or more pulse waves.

### TECHNICAL FIELD

The present invention relates to methods and apparatus for the non-invasive determination of blood pressure, and, more particularly, to the field of measuring pressure in blood vessels by applying pressure against the skin.

### BACKGROUND

Arterial blood pressure, a clinical vital sign comprised of systolic arterial blood pressure (hereinafter also referred to as SBP) and diastolic arterial blood pressure (hereinafter also referred to as DBP) is recognized as being of critical importance for monitoring, diagnosing, and treating a large number of diseases, as well as in normal health for assessment of cardiovascular fitness and other related conditions.

Due to longstanding tradition, the arterial blood pressure parameters are expressed and recorded using the non-metric unit of millimeters mercury (mmHg). This unit is utilized throughout this disclosure.

Measurement of arterial blood pressure can be performed either invasively or non-invasively.

Invasive methods require placement of an intra-arterial line (A-line), e.g., a cannula or catheter connected to a pressure sensor providing direct readings of both parameters. While this method is considered “gold standard” for 10-20% of high-risk patients such as those in intensive care units and during cardiovascular surgery, it cannot be used for routine monitoring and measurements due to its inherent risks including bleeding and infection.

The non-invasive methods are the de facto clinical standard for the absolute majority (>80%) of high-risk and all low-risk patients. These include the oscillometric method, and the auscultatory method, as well as several approaches that estimate pressure values without actually measuring them, such as estimations based on the pulse wave velocity (not applicable to the disclosed invention).

The oscillometric method is based on detection of the passage of pulse waves, essentially boluses of fluid, through a partially compressed arterial segment with said passage causing oscillations of pressure inside the inflatable elastic cuff. Such oscillations, their relation to the pressure in the cuff, as well as appearance and disappearance of oscillations as the pressure in the cuff changes, are then interpreted by an algorithm or a neural network and values for SBP and DBP are derived.

The auscultatory method is often considered preferable due to the following: (1) obtained values have been shown to be in close agreement with values obtained by the invasive method, and (2) as the operator listens to the sounds produced by turbulent blood flow, additional clinically-relevant signs may be evaluated such as unusual tone or character of the sounds, as well as the presence of the diagnostic sign known as auscultatory (acoustic) gap.

The auscultatory method utilizes the phenomenon known as Korotkoff sounds and the associated Korotkoff events. For the purposes of this disclosure a distinction is made between a Korotkoff sound (KS) and a Korotkoff event (KE) as defined below.

The mechanism of generation of Korotkoff sounds is typically cited as follows: while under normal conditions blood flow through an arterial segment is practically noiseless, an impediment, such as compression of the arterial segment, generates turbulent flow with numerous vortices and vibratory movements of the arterial wall producing a complex spectrum of sounds. Such impediment may be provided by compressing the arterial segment with an externally applied inflatable cuff, a rigid pressure applicator or even a fingertip.

As implemented in common clinical practice, the auscultatory method utilizes the Riva-Rocci sphygmomanometer, comprised of a pressure measurement device and an elastic inflatable cuff, applied around a limb to facilitate compression of the underlying vascular structures. Two anatomical locations are commonly used for placement of the inflatable cuff: (1) on the shoulder part of the upper limb proximally of the elbow and (2) around the distal forearm, at the wrist. Other placements of the cuff may include proximal or distal lower extremity; however, such placements are less common in current clinical practice.

While variations of the method are possible, clinical guidelines recommend the following steps: (1) inflating the cuff while checking the pulse distally of the cuff and noting the pressure at which the pulse disappears; (2) further inflating the cuff by to exceed the noted pressure by at least 10-15%; (3) slowly deflating the cuff while listening to Korotkoff sounds with a stethoscope placed distally of the cuff and recording pressures associated with specific sounds.

This sequence is considered standard of care and is used in assigning numbers to Korotkoff sounds (hereinafter also referred to as KS):

The 1st Korotkoff sound (1KS) is generated as the external pressure (applanation pressure) closely approximates but is less than the systolic arterial blood pressure causing the resuming of the blood flow through the arterial segment resumes but only during the passage of a pulse wave, as the arterial segment's lumen closes completely once the pulse wave passes. The turbulent flow through the arterial segment causes it to oscillate, which generates 1KS, typically described by human operators as “clear” and “tapping”, with the “tapping” component presenting with higher frequency sounds, reflecting the closure of the lumen between the pulse waves. Depending on the rate of deflation and the muscle tone in the limb, one or more pulse waves may produce the 1KS. In several clinical guidelines systolic arterial blood pressure is specifically defined as corresponding to the 1st Korotkoff event.

The 2nd Korotkoff sound (2KS) is generated as the externally applied pressure (applanation pressure) is less than the systolic arterial blood pressure and the lumen of the arterial segment is obturated by the factor less than 100% but greater than 50%-66% allowing for the blood flow through the arterial segment to persist, thus causing an easily discernible change in sound quality occurs: 2KS is both slightly longer in duration and is described as “swishing”, reflecting the preponderance of mid-range frequencies as the arterial segment's vibrations resemble that of a string more so than a cylinder or pipe.

The 3rd Korotkoff sound (3KS) is generated as the externally applied pressure (applanation pressure) is substantially below the systolic arterial blood pressure and the lumen of the arterial segment is obturated by the factor less than 50%-66%, allowing for the passage of a greater volume of blood with each pulse wave and the return to the normal speed of the flow. These changes cause a greater amount of kinetic energy to be dissipated and an increase in the number and variety of vibration modes that are now similar to the vibratory profile of a cylinder or a fluid-filled pipe. The associated 3KS is, therefore, more loud, slightly longer than 2KS, and is typically described as “crisp” indicating the appearance of higher frequency sounds.

The 4th Korotkoff sound (4KS) is the sound generated as the externally applied pressure (applanation pressure) closely approximates the diastolic arterial blood pressure, with minimal obturation of the arterial segment's lumen and minimal generation of turbulence during the passage of a pulse wave. Thus, 4KS occurs during the last pulse wave(s) passage that still produces some turbulence-generated sound, described as “whisper-like” but essentially similar in tone to the 3KS.

While there are 4 discernible types of Korotkoff sounds, the Korotkoff events (KE) are more numerous and their definitions vary. For the purposes of this disclosure 6 events are defined.

Events 1KE, 2KE, 3KE, and 4KE are defined as events during which the corresponding Korotkoff sounds 1KS, 2KS, 3KS, and 4KS are generated.

The 0th Korotkoff event (0KE) is a protracted silent event that precedes 1KE by one or several pulse wave(s) as the externally applied pressure is higher than the systolic arterial blood pressure and the arterial segment's lumen is obturated by nearly or exactly 100%. This event is not commonly used to in clinical practice but is of utility for the purposes of this disclosure as determination of 0KE allows for more precise detection of 1KE.

The 5th Korotkoff event (5KE) is a protracted silent event that occurs during the passage of unimpeded pulse waves that do not produce any turbulence and, thus, do not generate any discernible sound as the externally applied pressure is less than the diastolic arterial blood pressure. The first instance of 5KE as the externally applied pressure drops just barely below the diastolic blood pressure is a commonly defined event in clinical practice and medical research. In a properly performed measurement 4KE and 5KE differ by just one pulse wave and typically by less than 5 mmHg. Of note is the fact that it is also often traditionally described as the “5th sound,” even though this is the absence of detectable sound.

Table 1 presents the correspondence between KS and KE and externally applied pressure as it relates to SBP and DBP.

TABLE 1

| Applied pressure | | |
|---|---|---|
| boundaries | Korotkoff Sound | Korotkoff Event |
| P > SBP | None (background noise only) | 0KE |
| P ≅ SBP | 1 | 1KE |
| DBP << P < SBP | 2 | 2KE |
| DBP < P << SBP | 3 | 3KE |
| P ≅ DBP | 4 | 4KE |
| P < DBP | None (background noise only) | 5KE |

The aforementioned clinical phenomenon and diagnostic sign known as the auscultatory gap can only be ascertained utilizing the auscultatory method as it is defined as the drop of sound intensity or even complete disappearance of audible sounds, typically between 2KE and 3KE. The presence of auscultatory gap has diagnostic and prognostic significance: it is associated with higher risk of end-organ damage, and in systemic sclerosis it is correlated with the development of auto-antibodies to RNA polymerase and higher risk of renal and cardiovascular complications.

Several relevant issues are associated with the current guideline-compliant clinical measurements of arterial blood pressure.

“While coat hypertension” is the transient elevation of arterial blood pressure, typically by more than 10 mmHg, caused by the psychological stress and anxiety associated with the procedure, by the fact that the patient's body autonomy is challenged by another person, and by the discomfort caused by the cuff as it compresses the limb. This response to the measurement occurs in as many as 30% of patients and is reasonably reproducible.

As the inflated cuff causes all of the collateral arteries, peripheral nerves, and veins to be constricted, the patient's discomfort and even pain may be significant and may lead to muscle fasciculations, affecting the measurement, as well as refusal or premature termination of measurement.

During the measurement, there is substantial venous congestion in the limb from the moment the pressure in the cuff exceeds the peripheral venous pressure, with some research citing approximately 20 mm Hg for the upper limb, hence, even with minimal inflation, the cuff effectively cuts off venous drainage of the limb. Such venous congestion causes diminishment of Korotkoff sounds and interferes with the oscillometric method by reducing the pressure variance, leading to erroneous estimation of blood pressure.

The size of the cuff is also an important consideration: an oversized cuff causes underestimation of the pressure and a cuff that is too small causes overestimation. The American Heart Association recommends the use of four different sizes of the cuff for adult population and three different sizes for the pediatric population, which is also a potential source of error.

Recognition of these undesirable aspects, as well as the desire for facile, convenient and reliable monitoring of arterial blood pressure led to several attempts to improve the cuff-based methods (these are outside of the scope of this disclosure) and less numerous attempts to develop cuffless methods. The absolute majority of disclosures of cuffless methods relate to the oscillometric method and its variations.

It needs to be noted that oscillometric apparatus rely on a single stream of signal, that of pressure variance, require accumulation of large volumes of low-noise mechanical signal from a anatomical structure with multiple moving ligaments and muscles, a challenging if not prohibitively difficult task.

In addition, practically all of the currently disclosed cuff-free apparatus that apply and measure the applied pressure rely on actuators with a multitude of small precision-made parts that are subjected to significant forces causing deformation, wear, and subsequent need for replacement.

U.S. Pat. No. 5,941,828A for Hand-held non-invasive blood pressure measurement device, issued on Aug. 24, 1999 and assigned to Medwave, Inc., teaches a hand-held cuffless apparatus that is applied on top of an arterial segment and compresses said segment using a piston-like actuator. There are several unavoidable technical problems with such implementation, as the hand that holds the apparatus will need to be exceptionally steady. Per Newton's Third law, the action of the actuator on the underlying soft tissues will be met with the corresponding counteraction. To minimize the effects of said counteraction the hand holding the apparatus would need to be pushing it towards the arterial segment in a highly linear and steady fashion over a longer period of time.

U.S. Pat. No. 6,558,335B1 for Wrist-mounted blood pressure measurement device issued on May 6, 2000 and assigned to Medwave, Inc., is the further development of the device and method disclosed in U.S. Pat. No. 5,941,828A. The issue of the counteraction that causes the apparatus' piston to be pushed away from the soft tissues is addressed but not fully resolved by adding a rigid element, a hook that partially encircles the wrist while the actuator pushes against the soft tissue causing compression of the arterial segment. As the wrist is a complex anatomical structure, the movement and deformation of the wrist within the hook will generate the same need for an exceptionally steady application of force by the user that would have to increase and decrease in a near-linear fashion to obtain data suitable for analysis. As with other inventions assigned to Medwave, Inc., there is reliance on just one stream of data, that of pressure, being vulnerable to motion artifacts, noise and degradation of signal over the extended time needed for accumulation of the large volume of data sufficient for analysis.

Japan Patent JP4796025B2 for Pressure applying device and biological signal measuring device having the same issued on Oct. 19, 2011 and assigned to Samsung Electronics Co Ltd, teaches a wrist-based device that utilizes an elastic board with an actuator, thus making application of precise magnitude of pressure more reliable. Notably, the disclosure recognizes auscultatory method as the reference method for the oscillometric approach utilized in the disclosed method. No attempt is made to incorporate the reference method in the disclosed apparatus or method.

While it is desirable to be able to measure one's arterial blood pressure in a facile, intuitive, precise and validated manner, a cuffless device utilizing the oscillometric method for such purposes faces several technical problems, of which the most important are listed below.

First, using the oscillometric method would require measuring a small relative variation of volume that can be confounded by a simple movement of ligaments in the wrist as the volume and pressure change caused by such movement will be comparable and in some cases greater than the volume change due to passage of a pulse wave.

Second, compression and slow release of the arterial segment with a cuff, inflatable bubble or an actuator requires substantial energy expenditures, such as provided by a rechargeable battery needing charging and maintenance, being prone to aging and loss of capacity, as well as containing reactive chemical compounds.

Third, a miniaturized oscillometric device will be built from small, precision made moving parts subject to significant mechanical forces, deformation and wear, increasing the cost and likelihood of failure and need to repair.

Fourth, using only one kind of data, namely, variation of pressure in the typical 0-200 mmHg (0-3.87 psi) limits the amount of interpretable information that can be extracted from such minimal dataset.

Fifth, monitoring of BP by algorithmic analysis of pressure variation data requires a tight bracelet/mounting strip that reduces the user's comfort and motivation to perform regular measurements.

Alternative methods, such as estimation of blood pressure based on pulse wave velocity also suffer from at least one technical and one methodological problem.

The technical issue, is the fact that the PWV method relies on a calculations that introduce at least one person-specific and mutable over time correction coefficient that needs to be initially quantified and adjusted or remeasured on a regular basis as the condition of the user's arteries changes due to aging, levels of hydration, atherosclerosis, additionally confounded by the fact that the correction coefficients are in a non-linear relation both to heart rate and the arterial pressure itself;

The methodological issue is the absence of clinical compatibility: healthcare professionals in the USA and other countries use the oscillometric method or the auscultatory method (or both) but no PWV-based apparatus is in wide use and techniques of PWV-based measurements are, at best, unfamiliar.

The solution to these and related technical challenges is a cuffless apparatus utilizing the auscultatory method with applanation pressure provided by the action of the user's finger in the manner similar to pulse taking and self-validating two-sensor method of data interpretation.

The set of technical problems the disclosed invention needs to resolve includes several apparatus-related and several user-related issues.

The oscillometric method requires measuring a small relative variation of volume that can be confounded by a simple movement of ligaments in the wrist as the volume and pressure change caused by such movement will be comparable and in some cases greater than the volume change due to passage of a pulse wave.

Cuffless apparatus that utilize a mechanical actuator to provide the necessary applanation force onto the arterial segment require precision-made moving parts that have to withstand repeated application of substantial pressure (typically up to 250 mmHg, approximately 4.8 psi). Such moving parts are subject to wear and misalignment; they are also more expensive to manufacture and maintain.

The action of mechanical actuators requires significant energy, typically provided by an electric battery that necessitates charging, may fail during measurement and is subject to unavoidable loss of capacity over time.

Measurement of variations of volume of a short arterial segment necessitates slow and linear change in the magnitude of the applanation force that is difficult to achieve given the movement of ligaments and contractile activity of muscles, thus requiring mechanical actuators.

A handheld actuator requires an exceptionally steady positioning that is difficult to maintain, since as the actuator produces the applanation force, the hand holding it will have to counteract the equal and opposite reaction.

Cuffless apparatus that utilize the oscillometric method cannot supply certain kinds of valuable clinical information: the auscultatory gap cannot be ascertained even in principle as it is an acoustic phenomenon.

Methods based on estimation of blood pressure based on indirect measurements, such as based on pulse wave velocity are unreliable as pulse wave velocity changes not only in relation to blood pressure but also in relation to the condition of the blood vessel, such as atherosclerosis and age-related diminishment of elasticity.

In many disclosed inventions the user remains a passive participant in the measurement and the related stresses and anxieties causative of “white coat hypertension” are not detected, not addressed and not compensated for.

In case of measurements based on the oscillometric method, the user is aware that the method utilized is different from the methods most commonly used by physicians and other health care professionals, i.e., the auscultatory method, which causes the user to question reliability and applicability of the measurement.

### SUMMARY OF INVENTION

The essence of the disclosed invention that addresses the aforementioned technical and user-related problems can be formulated as follows:

(1) Measurement of arterial blood pressure and detection of auscultatory gap are based on the clinical standard, namely, detection of Korotkoff sounds and corresponding Korotkoff events, thus directly compatible with measurements performed by clinical personnel;

(2) Applanation force is applied onto the arterial segment by the user's hand, thus negating the need for a mechanical actuator and high-capacity power supply;

(3) The user's hand or finger applies the applanation force in a natural pulsatile manner with the compression of the arterial segment lasting less than 10-12 pulse waves while not requiring the user to maintain a slow near-linear change in the magnitude of the applanation force;

(4) The user is guided by the apparatus with simple to understand means such as voice prompts or sound prompts including the optional real-time replay of the detected Korotkoff sounds; and, in some embodiments, messages and images displayed on the optional screen(s) of the apparatus;

(5) Korotkoff sounds and corresponding Korotkoff events KE1 and KE4 are rapidly identified by an algorithm based on changes in sound amplitude transitioning from sound being present to sound being absent, thus allowing to obtain estimated values for SBP and DBP with said estimated values being used to adjust the instructions for the user involving magnitude of applied pressure, duration of ramping up and ramping down of the applied pressure, and direction of the vector of application of pressure;

(6) During the self-validating portion of the measurement, as the user follows the adjusted instructions based on the estimated values of SBP and DBP, the pressure and sound field datasets are subjected to machine learning recognition of Korotkoff events according to two different modalities to facilitate self-validation interpreted as less than a predetermined amount of disagreement between values of SBP and DBP obtained by first modality and values of SBP and DBP obtained by the second modality;

(6.1) The first modality utilized for the self-validating portion of the measurement being the machine learning assessment and near-real-time identification of Korotkoff events from K0 to K5 as well as the auscultatory gap in the image that is the graphical representation of the pressure signal overlayed over the sound spectrogram, that is, a graphical representation of sound amplitude as function of frequency with time as parameter; noting that machine learning setups (neural engines) are capable of excellent results in recognition of complex graphical images.

(6.2) The second modality utilized for the self-validating portion of the measurement being the machine learning assessment and near-real-time identification of Korotkoff events, namely 1KE, 2KE, 3KE, and 4KE, as well as the auscultatory gap, in the dataset comprised of datapoints of pressure as function of time and sound field as function of time; with the machine learning setup (neural engine) detecting the correlation between the first derivative (slope) of the pressure as function of time and the first derivative (slope) of the sound amplitude as a function of time.

(7) The user is an active participant of the measurement as the user applies the applanation force in a fashion similar to the familiar and non-threatening procedure of pulse-taking while interacting with the apparatus, which reduces the stresses and anxieties associated with the “white coat hypertension”.

(8) As only one arterial segment is compressed, neither the discomfort due to interruption of blood flow through the downstream circulation, nor the venous congestion are present during the measurement;

(9) Existing devices such as smart watches and smart phones can be utilized for data acquisition and processing as such devices commonly possess sufficient computational capacity and built-in sensors;

(10) As currently available pressure and sound sensors are characterized by high sampling rates combined with very low power requirements, they can be powered by a long-lasting or disposable battery, by an induction coil or a wireless antenna converting wifi or Bluetooth signals into electromotive force.

### BRIEF DESCRIPTION OF THE DRAWINGS

FIG. 1A illustrates an overview of the disclosed apparatus attached to user's wrist according to some embodiments of the present disclosure wherein the applanation force is generated by the user's finger;

FIG. 1B shows the schematic representation of the human wrist with the arterial segment compressed by the applanation force generated by the user's finger and transmitted by the disclosed apparatus to the arterial segment;

FIG. 2 shows the schematic representation of the electronic components of the disclosed apparatus;

FIG. 3A shows the sequence of clinical phenomena known as Korotkoff events (KE) and Korotkoff sounds (KS) overlayed onto curves representing sound amplitude and applied pressure as functions of time.

FIG. 3B illustrates a partial representation of tangents to said curves at select KE and their slopes expressed in radians;

FIG. 4A shows the experimentally obtained dataset consisting of pressure magnitude and pulse waves as functions of time overlayed with Korotkoff events;

FIG. 4B shows the experimentally obtained dataset consisting of pressure magnitude and sound frequency maps as functions of time and represented as a graphical image with Korotkoff sounds and Korotkoff events indicated;

FIG. 5 illustrates another exemplary device according to some embodiments of the present disclosure wherein the pressure applicator is a separate assembly and the processing unit is a smart watch;

FIG. 6 shows the exemplary device of FIG. 5 from the front, left, and top sides with two schematic insets to illustrate arrangement and connections of the internal components;

FIG. 7A illustrates a pressure applicator and processing unit as a part of an exemplary setup according to some embodiments of the present disclosure wherein the processing unit supplies energy to the pressure applicator's sensors and circuitry via induction coils;

FIG. 7B shows a schematic inset to illustrate arrangement and connections of the internal components of the pressure applicator of FIG. 6A;

FIG. 8A illustrates an exemplary setup according to some embodiments of the present disclosure wherein the pressure applicator lacks sensors and is connected to the sensors built into the processing unit with a pressure-and-sound guide

FIG. 8B shows a schematic inset to illustrate arrangement and connections of the internal components of the exemplary setup of FIG. 8A;

FIG. 9 illustrates the sequence of steps of an exemplary method for measurement of blood pressure and assessment of presence of the clinical phenomenon known as the auscultatory gap relating to initialization, initial setup and calibration of the hardware;

FIG. 10 illustrates the sequence of steps of an exemplary method for measurement of blood pressure and assessment of presence of the clinical phenomenon known as the auscultatory gap consisting of the initial estimation of values, and two separate machine learning analyses for the more precise measurement;

FIG. 11 illustrates the sequence of steps of an exemplary method for measurement of blood pressure and assessment of presence of the clinical phenomenon known as the auscultatory gap relating to the post-measurement processing and termination of the sequence.

### DETAILED DESCRIPTION

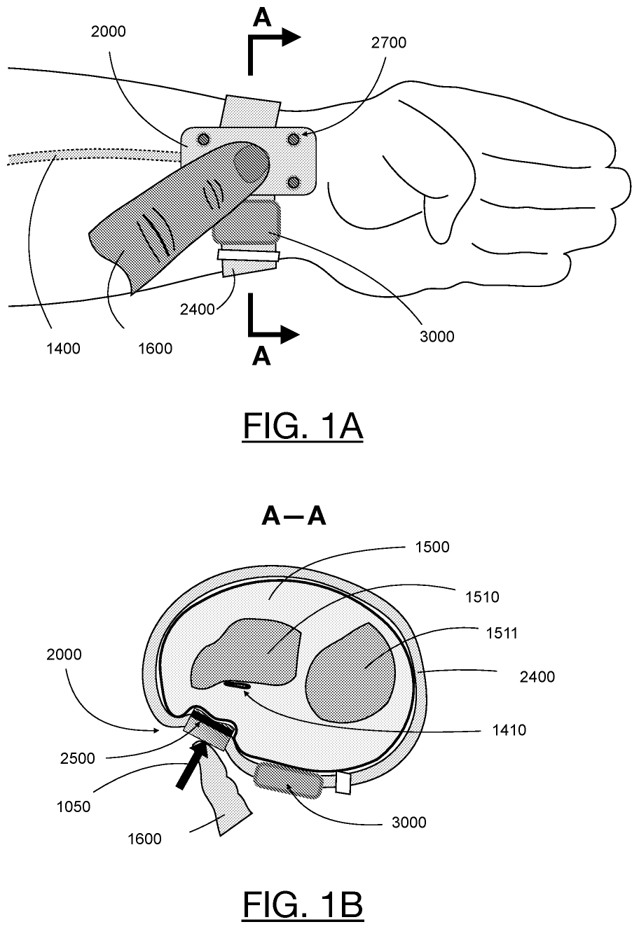

FIG. 1A depicts an embodiment of the disclosed invention comprised of the pressure applicator 2000 fastened to the wrist of the user with the holding strap 2400 with the first surface of the pressure applicator 2000 adjacent to the user's wrist in the immediate proximity of the arterial segment (radial artery) 1400, while the user's finger 1600 applies mechanical force onto the pressure applicator and distributed over the first surface of the pressure applicator causing impeded blood flow through the artery and generation of Korotkoff sounds. Processing unit 3000 is depicted as attached to the holding strap 2400.

Four pressure gauges 2700 are embedded into the pressure applicator 2000 serving to allow the user verify the correct application of mechanical force as these are activated and change appearance to alert the user as the pressure reaches predetermined levels. In some embodiments of the disclosed invention the pressure gauges 2700 are calibrated to change appearance as the pressure levels reach 60, 100, 140, and 180 mmHg. Cross-section through the setup is indicated with arrows (1-1).

FIG. 1B depicts the schematic inset to illustrate arrangement and connections of the internal components along the A-A line as indicated on FIG. 1A and presents the disclosed invention in the process of measurement of arterial blood pressure: mechanical force 1050 applied by the user's finger 1600 onto the pressure applicator 2000 and further transferred through the soft tissue 1500 of the wrist acting on the arterial segment causing its compression against the radius bone 1510 with such compression generating Korotkoff sounds due to impeded blood flow through the compressed arterial segment 1410. Processing Unit 3000, ulna (bone) 1511, soft tissue 1500, and holding strap 2400 are depicted for reference purposes only.

The schematic of the pressure applicator 2000 reveals rigid delineators 2500 facilitating the immobilization of the arterial segment to minimize axial deviations and uneven distribution of pressure;

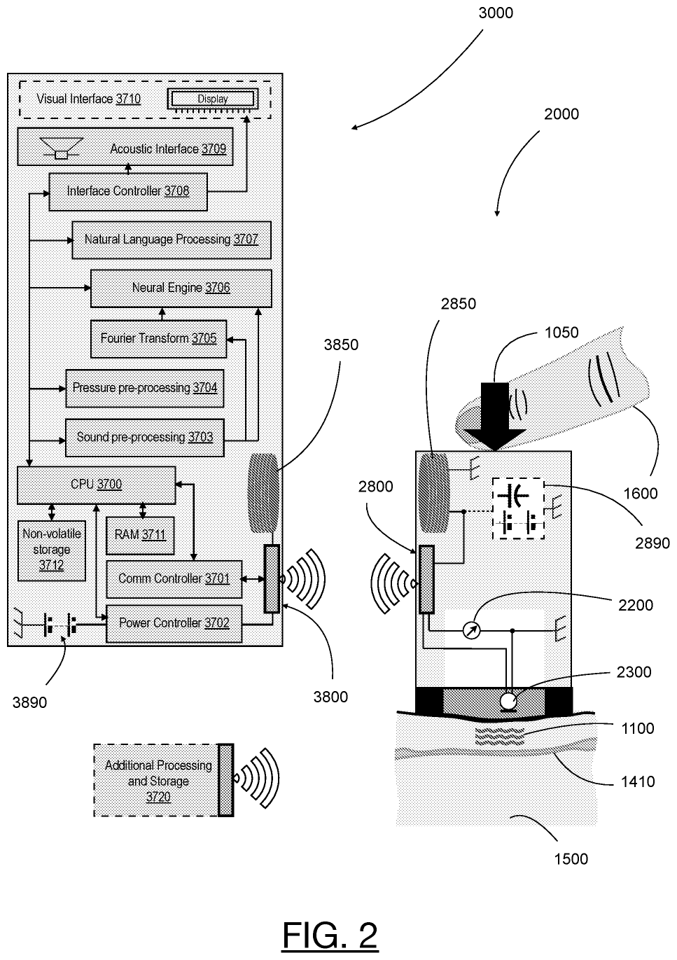

FIG. 2 presents the schematic depiction of an embodiment of the disclosed invention in greater detail and in a functional relation. The depicted embodiment is comprised of the pressure applicator 2000 and the processing unit 3000 in a functional wireless communication link facilitated by pressure applicator's wireless setup 2800 and processing unit wireless setup 3800.

Pressure applicator 2000 is equipped with the pressure sensor 2200 and sound sensor 2300 in functional connection with the wireless setup 2800. Given the very low power requirements of the sensors, electrical power may be supplied in real time as power-over-wireless, either IEEE 802.11 (Wi-Fi) or IEEE 802.15.1 (Bluetooth); or, optionally, by the pressure applicator induction coil 2850. Electrical charge may be optionally stored in a power source 2890 comprised of condensers or rechargeable power cells.

Prior to initiation of measurement pressure applicator 2000 is positioned on the user's body over a selected artery, commonly, the wrist above the radial artery. During measurement user applies force 1050 with their finger 1600 that compresses the soft tissue 1500 and the arterial segment 1410, with said compression generating Korotkoff events 1100. Pressure sensor 2200 and sound sensor 2300 collect data as time-function of pressure dataset Pt and time-function of the sound field dataset SFt; these data are transmitted in parallel in real time to the processing unit 3000.

Pressure magnitude values are captured as millimeters mercury, a non-standard but universally used clinical unit of measurement, by the pressure sensor 2200 with the sampling rate typically capped at 1000 Hz. Sound field parameters are captured by the sound sensor 2300 with the sampling rate typically capped at 1000 Hz. These data are transmitted to the processing unit 3000 for further processing.

Processing unit 3000 is comprised of the main processor 3700 facilitating computational and data processing operations, random access memory (RAM) 3711, and non-volatile storage 3712 providing correspondingly short- and long-term storage of data.

Electrical power is supplied by the processing unit power source 3890 and controlled by the power controller 3702 that supplies power to the CPU 3700, as well as processing unit wireless setup 3800 and the processing unit induction coil 3850; wireless communication is facilitated by the comm controller 3701 and processing unit's wireless setup 3800 in functional communication with the pressure applicator 2000 and, optionally, other setups.

The main processor is connected to secondary setups including sound pre-processing setup 3703, pressure pre-processing setup 3704, Fourier transform setup 3705, neural engine 3706, natural language processing setup 3707, and interface controller 3708. The interface controller is further connected to the acoustic interface 3709 and the optional visual interface 3710 comprised of a hardware controller and a display.

In some embodiments of the disclosed invention the main processor, memory and some or all of the controllers and setups may be combined as a system on a chip (SoC).

Pressure magnitude data is supplied into the pressure pre-processing 3704 for signal enhancement and noise reduction as necessary, calculation of average heart rate (aHR) parameter and the heart rate variance (vHR) parameter, as well as optional pre-processing such as averaging, segmentation, and integration for incorporation into datasets for analysis.

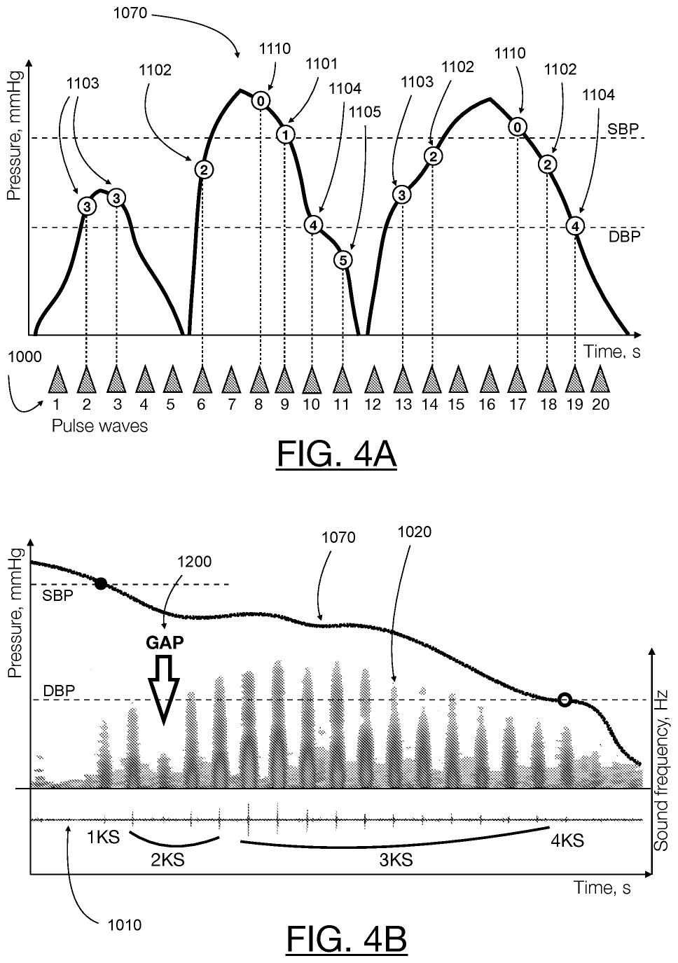

Sound field data (essentially small variance of the pressure parameter with its spatial and temporal distribution) is processed by the sound pre-processing 3703 and Fourier transform setup 3705 into a numerical dataset containing integrated sound amplitude values as a function of time and a sound frequency-amplitude spectral map with optional further transform into a graphical image as a spectrogram wherein the horizontal axis is parametric and relates to elapsed time, vertical axis is sound frequency and the relative amplitude of a specific characteristic frequency is represented by grayscale density or pseudocolor. An example of a sound spectrogram is presented as FIG. 4B reference sign 1020.

The improvement herein is due to the fact that a neural engine 3706 can be trained to process two kinds of data: (1) numerical dataset components recognized as Korotkoff events (KE) and the auscultatory gap phenomenon based on the relation between pressure magnitude and amplitude of pressure-generated sound; and (2) the graphical image of the spectrogram can be segmented into regions of the spectrogram by the neural network so that these image regions can be recognized as Korotkoff events and the auscultatory gap by the well-established image recognition machine learning setups.

The ability to correlate and contrast two separate results obtained by radically different methods of interpretation allows for self-validation and a measurement that is more precise and less affected by noise and low quality data.

In some embodiments of the disclosed invention accumulated data, reports, recommendations and user responses may be further processed and stored off-site in additional processing and storage unit 3720.

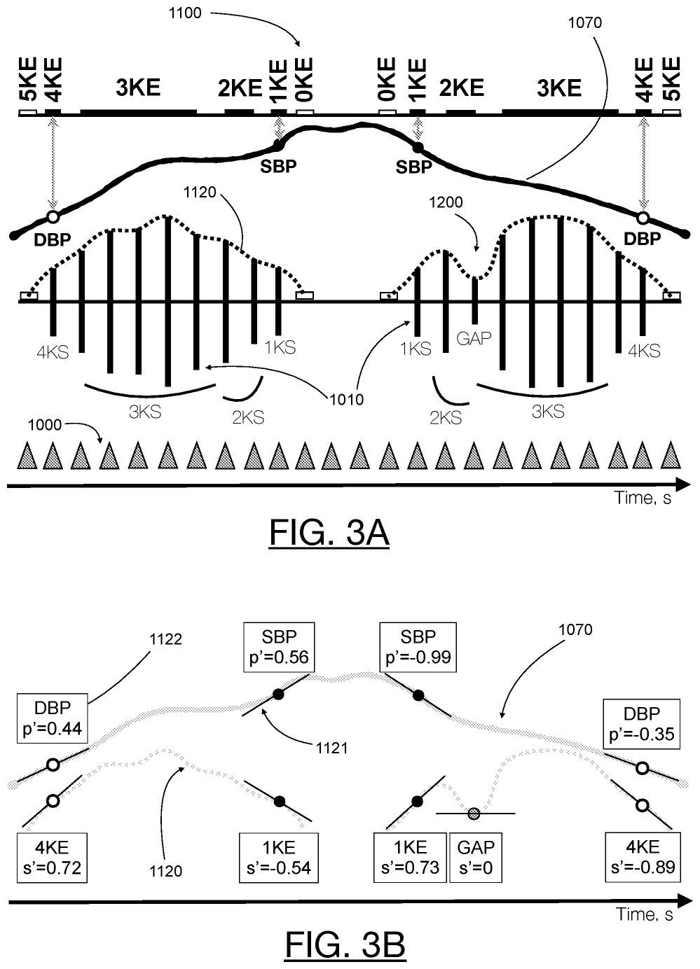

FIG. 3A schematically depicts the sequence of Korotkoff Events (KE) 1100 and the auscultatory gap (GAP) 1200, with KE and GAP being superimposed onto the curves representing applied pressure 1070, sound amplitude 1010 as functions of time with pulse waves 1000 illustrated in a simplified fashion.

As the user applies progressively increasing pressure 1070, the initial clinically-relevant Korotkoff Event (KE) is the 5KE, defined as the last pulse wave that produces no sound under increasing pressure. As the applied pressure becomes near-equal to the Diastolic Blood Pressure (DBP), the characteristic 4KS is recorded corresponding to 4KE.

As the pressure increases, properties of Korotkoff sounds change, allowing for identification of 2KS and 3KS and the corresponding protracted 2KE and 3KE. As the applied pressure increases further and becomes near-equal to the Systolic Blood Pressure, the last recorded sound before the lumen of the arterial segment is fully closed is 1KS, corresponding to 1KE, and the following pulse wave fails to overcome the applied pressure, causing absence of sound, recognized as 0KE.

Following 0KE one or more pulse waves may generate no sound and are not recognized as Korotkoff events. As the user begins to reduce the applied pressure, KS1 is generated when the pressure is just slightly (typically, 5-10 mmHg) below the Systolic Blood Pressure (SBP), with this first appearance of sound being recognized as KE1 and the pulse wave immediately preceding KE1 is recognized as KE0.

Further reduction of applied pressure causes changes in the character of KS, with KS2 and KS3 following in succession and being recognized as protracted KE2 and KE3. As the applied pressure declines further, the last recorded sound is KS4 as the pressure is near-equal to the Diastolic Blood Pressure, and the next pulse wave that fails to produce KS is recognized as 5KE.

Notably, for the purposes of subsequent analysis, sound amplitude as a function of time may be represented as a curve 1120 fitted to the amplitudes of Korotkoff sounds 1010.

FIG. 3B illustrates one of the methods of analysis of the obtained curves, namely pressure as a function of time 1070 and sound amplitude as a function of time 1120 is via differentiation, essentially by determining the slope, expressed in radians, of tangent lines 1121 drawn to the pressure curve and the sound amplitude curve.

As time passes, these derivatives, indicated as p′ for the pressure curve and s′ for the sound amplitude curve, will take specific values 1122, represented for calculation and conversion convenience as contained in the [−π/2; π/2] bracket, or, approximately [−1.5708; 1.5708] with the negative values indicating the curve going downward and a zero value indicating the tangent being parallel to the timeline.

Preliminary detection of events such as 1KE, 4KE and GAP is accomplished by establishing a correlation between a near-constant value (positive or negative) of p′ indicating steady increase or decrease in applied pressure and a sudden change of the value of s′ from near-zero to a positive value or from a negative value to a near-zero.

In this step the 4KE is detected either when p′ is positive and s′ undergoes change from near-zero value to a positive value or when p′ is negative and s′ undergoes change from negative to near-zero.

The 1KE is detected either when p′ is positive and s′ undergoes change from negative to near-zero value or when p′ is negative and s′ undergoes change from near-zero to positive value.

GAP is detected in the more complex case (only one instance illustrated) when p′ is negative and s′ changes from negative to near-zero to positive with the GAP point being when s′ is near-zero and the point is located between 1KE and 4KE. Alternatively, GAP is detected when p′ is positive and the s′ changes from negative to near-zero to positive with the GAP point being when s′ is near-zero and the point is located between 4KE and 1KE.

In some of the embodiments of the current invention analysis of the slopes can be performed algorithmically or, in other embodiments, it can be performed by a machine learning setup within a neural engine or a similar processor.

FIG. 4A presents the sequence of Korotkoff events (KE) as detected during a measurement performed with a pulsatile application of pressure by the user according to the method of the current disclosure. Applied pressure 1070 is presented as a function of time and pulse waves 1000 are superimposed on the graph and numbered from 1 to 20.

Three attempts at application of pressure are presented.

Of note is the fact that many users will have difficulty generating with their fingers a sequence of slowly and steadily increasing and decreasing pressure, thus the integral part of the disclosed invention is allowing the user to apply pressure as several attempts, in a pulsatile fashion.

During the first attempt user-applied pressure exceeds the true value of the diastolic blood pressure (DBP) but does not exceed the true value of the systolic blood pressure (SBP). As a result KE3 is detected 1103 corresponding to pulse waves 2 and 3.

The obtained value for applied pressure corresponding to KE3 is used to adjust the instructions provided to the user, specifically to increase the peak pressure applied to the pressure applicator and to increase and decrease the pressure more slowly. The user complies with the adjusted instructions during the second and third attempts successfully.

During the second attempt, the applied pressure rapidly exceeds the DBP and so only one KE is recorded 1102, being KE2 during pulse wave 6. As the user begins to reduce the applied pressure, KE0 is detected 1110 as absence of sound at pulse wave 8 and the more clinically important KE1 is recorded 1101 during pulse wave 9 as the pulse wave arrives as the applied pressure is near-equal to the true value of SBP.

As the user continues to reduce the applied pressure, KE4 is recorded 1104 during pulse wave 10 as the applied pressure is near-equal to DBP and KE5 is detected 1105 as the absence of sound during pulse wave 11.

Instructions for the user are again adjusted taking in consideration the obtained values for SBP and DBP as well as the pressure values corresponding to KE0 and KE5, establishing the brackets for applied pressure when it should be changed more slowly to allow for the more precise measurement or for verification of SBP and DBP values obtained during the second attempt.

During the third attempt the user tries to increase and decrease the applied pressure more slowly; however, in reality the applied pressure rapidly increases so that KE3 is recorded 1103 during pulse wave 13 and KE2 is recorded 1102 during pulse wave 14.

As the user-applied pressure begins to decline, KE0 is detected 1110 during pulse wave 17 when the applied pressure is no more than 5% in excess of the SBP as obtained during the second attempt, and KE2 is recorded during pulse wave 17, hence confirming the value of SBP with acceptable accuracy.

The final event is KE4 recorded 1104 during pulse wave 19 and as the applied pressure is almost exactly the same as the value for DBP obtained in the second attempt, the values are confirmed and the measurement is stopped.

FIG. 4B depicts the successful detection of the auscultatory (acoustic) gap phenomenon during a measurement performed according to the disclosed method. Depicted is the trace for pressure magnitude 1070 recorded as a function of time.

As the user-applied pressure declines, the sequence of KS is detected and recorded both as amplitude 1010 and frequency map (spectrogram) 1020; with both superimposed on the graph. Following the first instance of the 2KS there is a more than 70% drop in amplitude of the KS and a significant change in its spectrum (disappearance of the higher harmonics) with the next KS being of the normal (expected) amplitude and spectrum.

The rest of the recording is non-contributory as the normal (expected) sequence of 3KS and 4KS is observed. The drop in amplitude and change of the spectrum of the 2KS allows to establish the presence of the auscultatory (acoustic) gap phenomenon 1200, associated with such clinical diagnosis as systemic sclerosis but also indicative of presence of atherosclerotic plaque and hemodynamic abnormalities.

This clinically-relevant information cannot be obtained with the oscillometric methods of blood pressure measurement and constitutes one of the advantages of the disclosed invention.

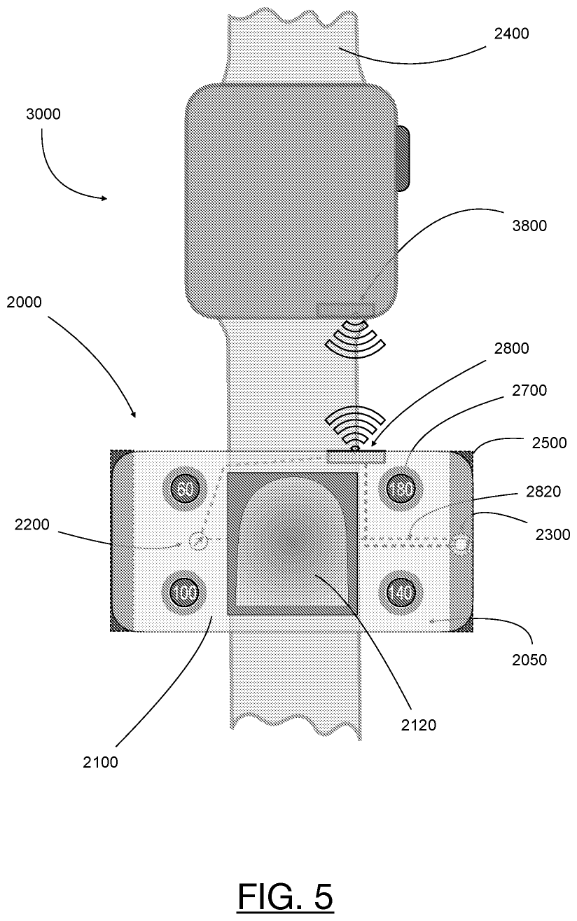

FIG. 5 depicts an embodiment of the disclosed invention in which the pressure applicator 2000 and the processing unit 3000 are attached to the holding strap (bracelet) 2400 and not stacked.

The pressure applicator 2000 is equipped with a wireless setup 2800 in a functional data connection to the processing unit 3000 that is also equipped with a wireless setup 3800 that facilitate a functional wireless connection as well as delivery of power via wifi/Bluetooth to the pressure sensor 2200 and the microphone 2300.

Of note is that the current state of electronic component optimization allows for manufacturing of sensors that require very low power while the specification for Bluetooth Class 3 (lowest permitted power) lists the power bracket as up to 1 mW. Some of the readily available microphones (sound sensors) such as TDK T5818 (InvenSense, San Jose, CA) operate between 0.130 mA and 0.33 mA at 1.8 V, thus requiring approximately 0.23 to 0.6 mW.

Similarly low-powered pressure sensors are also readily available making it possible to operate the pressure applicator sensor circuitry without a battery with all power supplied by the wireless antenna. In some embodiments of the disclosed invention an energy storage device may be employed such as a rechargeable battery or a condenser (not shown).

The use of wireless transmission as a source of energy is one of the distinctive features of the embodiment of the disclosed invention depicted in FIG. 5.

In addition to the pressure sensor 2200, sound sensor 2300 connected to the pressure applicator's signal bus 2820 and the wireless setup 2800, the pressure applicator 2000 has two rigid delineators 2500 and pressure equalizing elastic material 2050 that together comprise the first surface o the pressure applicator 2000; while the second surface has one or more pressure gauges 2700. The function of the pressure gauges is identical to the function described in relation to FIG. 4B.

The second surface of the pressure applicator 2100 also has the rigid concave support 2120 for the user's finger whereupon the user applies force to cause compression of the arterial segment (not shown). The presence of the rigid concave support 2120 differentiates this embodiment of the disclosed invention from the embodiment depicted in FIG. 4 as the rigid concave support in addition to serving as the point of application of pressure by the user also serves as a source of sound for sound sensor calibration.

As a rigid object of defined dimensions and density the rigid concave support 2120 when tapped with another rigid object produces sounds of defined frequency and amplitude that are used for the initial calibration of the sound sensor and for periodic maintenance. In some of the embodiments of the disclosed invention the tapping/striking object may be the user's fingernail or a common object such as a pencil.

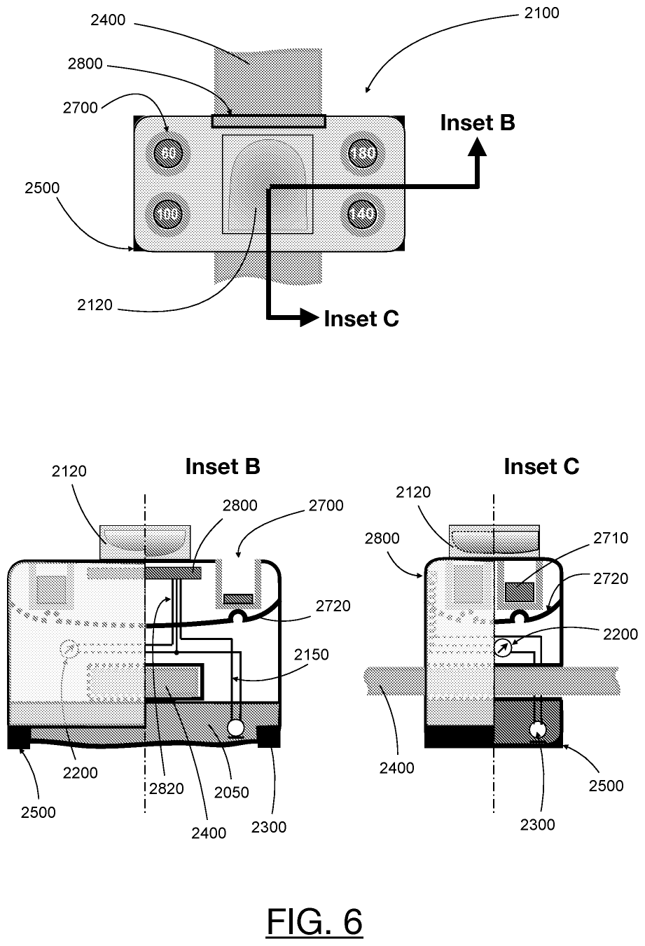

FIG. 6 presents the top, left and front views of the pressure applicator 2000 according to one of the embodiments of the disclosed invention, including the embodiment depicted in FIG. 5. Partial schematic insets to illustrate arrangement and connections of the internal components are indicated on the top view as Inset B (in relation to the front view) and Inset C (in relation to left view).

The top view of the pressure applicator is essentially that of its second surface 2100 with the rigid concave support 2120 whereupon the user's finger may apply force sufficient to generate pressure to compress the arterial segment underneath (not shown). Several pressure gauges 2700 are present, calibrated to change appearance at pressures equal to 60, 100, 140 and 180 mmHg and intended to alert the user to the level of pressure to be achieved or maintained. The pressure applicator is fastened to the holding strap (bracelet) 2400 that passes through the internal cavity of the pressure applicator.

The front view combined with Inset B reveals the relative placement of such parts as the rigid delineators 2500 and pressure equalizing elastic material 2050 as well as the profile of the rigid concave support 2120 as seen from the front of the pressure applicator and in the cross-section.

The internal elements of the pressure applicator are revealed in the Inset B: the internal cavity 2150 and electronic components such as the pressure sensor 2200, the sound sensor 2300, the pressure applicator's signal bus 2820, and the wireless setup 2800. The holding strap (bracelet) 2400 passes through the internal cavity 2150 facilitating minimal positional deviations laterally and longitudinally.

The left view combined with Inset C reveals the relative placement of the components of the pressure applicator such as the sound sensor 2300 embedded in the rigid delineator 2500 as well as a detailed view of the elements of the pressure gauge 2700 such as the elastic element 2720, pressure gauge mounting 2730, and the visual indicator 2710.

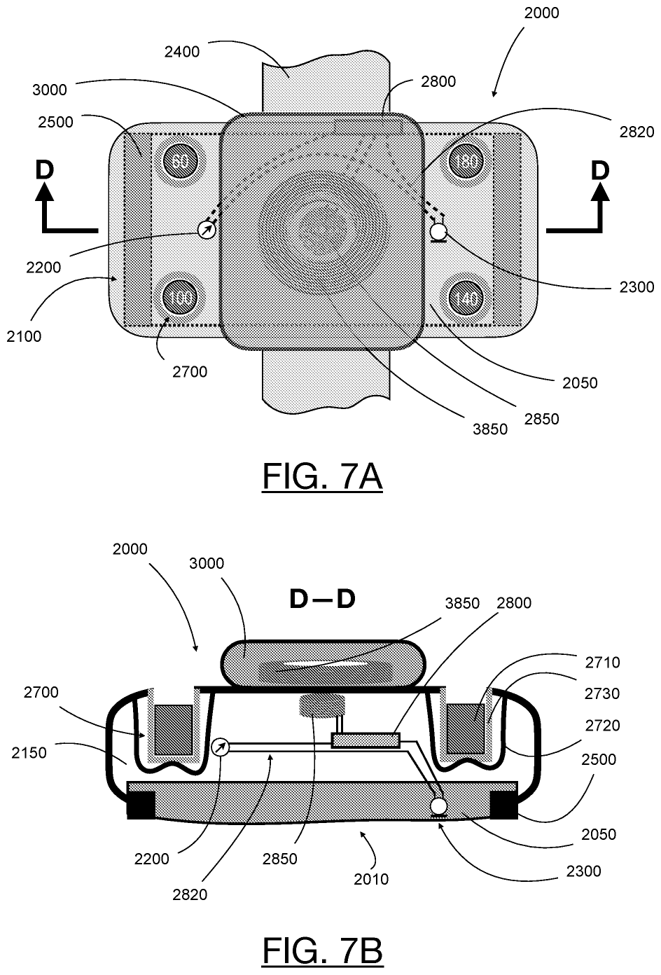

FIGS. 7A and 7B depict one of the embodiments of the disclosed invention in which the pressure applicator 2000 and the processing unit are stacked on top of each other with the induction coil of the pressure applicator 2850 and the induction coil of the processing unit 3850 being in close proximity, allowing for the generation of electromotive force and supply of electrical energy to the components of the pressure applicator 2000.

FIGS. 7A and 7B also present the constituent parts of the pressure applicator 2000 as seen from the top (FIG. 6A) and partially occluded by the processing unit 3000. The pressure applicator 2000 has two surfaces and an internal cavity (shown on 6B), with the first surface 2010 (shown on 6B) intended to transfer user-applied pressure to the soft tissues of the user's wrist (not shown) and the second surface 2100 used for coupling with the processing unit 3000 as well as to provide visual indication of the pressure applied as a function of pressure gauge setups 2700.

The first surface has two rigid delineators 2500 determining the length of the arterial segment used for the arterial blood pressure measurement and also stabilizing the pressure applicator against the wrist of the user thus reducing the lateral shifting. Between the rigid delineators 2500 the first surface is comprised of pressure-equalizing elastic material 2050, preferably a biocompatible gel serving to further reduce the unevenness of application of pressure in the longitudinal and lateral directions.

A sound sensor 2300 such as a microphone or sound transducer is embedded in the first surface 2010. In some embodiments of the disclosed invention a sound sensor array of two or more sound sensors may be used to improve sensitivity and obtain a plurality of measurements that are spatially distributed.

The internal cavity 2150 contains a pressure sensor 2200, as well as pressure applicator's signal bus 2820 comprised of signal processing and transmission circuitry connected to the induction coil of the pressure applicator 2850, the pressure sensor 2200, the sound sensor 2300 and to the pressure applicator's wireless setup 2800 in a working connection to the processing unit 3000.

The processing unit 3000 is stacked on top of the pressure applicator 2000 in close proximity to the second surface of the pressure applicator 2100. It is fastened to the user's wrist by the holding strap 2400 with an optional buckle or other fastener. The holding strap serves to position the assembly of 2000 and 3000 over the desired segment of the user's wrist and also reduces longitudinal and lateral shifts as well as rotational displacement during application of pressure by the user.

FIG. 7B depicts the schematic inset D-D to illustrate arrangement and connections of the internal components of the assembly of the pressure applicator 2000 and processing unit 3000 as presented in FIG. 7A. The internal cavity 2150 and the pressure gauges 2700 are more clearly explicated.

The internal cavity 2150 contains a pressure sensor 2200, as well as pressure applicator's signal bus 2820 comprised of signal processing and transmission circuitry connected to both the pressure sensor 2200 and the sound sensor 2300 and to the pressure applicator's wireless setup 2800 in a working connection to the processing unit 3000.

The internal cavity 2150 also houses one or more (two depicted) pressure gauge setups 2700 comprised of a visual indicator 2710 in a mounting 2730 that is actuated by the elastic element 2720 such as an elastic membrane or a spring. As the user applies pressure onto the pressure applicator 2000 the elastic element 2720 is compressed and actuates the visual indicator 2710 causing it to change appearance such as extend outside the second surface 2100.

In some of the embodiments of the disclosed invention there may be four pressure gauges 2700 capable of changing their appearance such as by extending outside the second surface 2100, by color change or other similar means.

These gauges may be calibrated to alert the user that the pressure in the internal cavity equals 60, 100, 140, and 180 mmHg or in other embodiments different values may be chosen such as the desirable (normal) upper value for DBP, namely 80 mmHg and the desirable (normal) upper value of SBP, namely 120 mmHg. These values correspond to the American Heart Association's guidelines for Americans as published in 2017.

Additionally, abnormal pressure values may be chosen to be indicated by the pressure gauges, such as 50 mmHg, an abnormally low (hypotensive) blood pressure and 140 mmHg, an abnormally high blood pressure corresponding to the SBP of stage 2 hypertension.

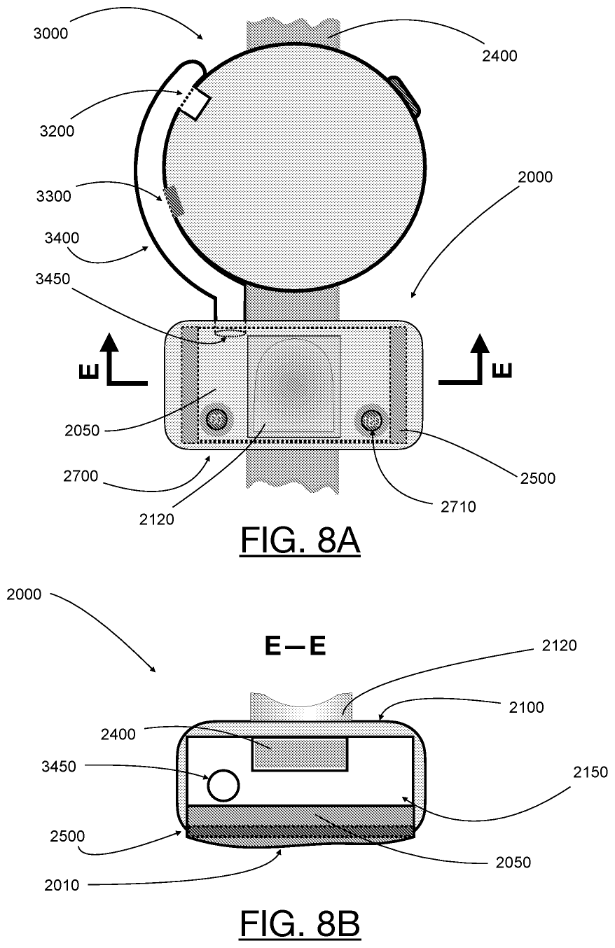

FIGS. 8A and 8B depict another embodiment of the disclosed invention wherein the processing unit 3000 contains both the pressure sensor and the sound sensor. Currently available wrist-worn devices such as smartwatches often possess sensors to facilitate measurement of barometric pressure and to provide with voice communication capability. Some of said smartwatches also have pressure sensors built into their screens to provide access to different commands of the operational system in the form of ‘smart menus’.

Additionally, accelerometers may be present in ‘smart devices’ for the purposes of monitoring movement and exercise. Acceleration data can be utilized to calculate applied force and dividing the applied force over the surface area of the pressure applicator allows for assessment of pressure.

The disclosed invention may be utilized in conjunction with such readily available wrist-worn devices that are repurposed as processing units for the disclosed invention, or the processing unit may be specifically and solely designed and manufactured for the purposes of measurement of arterial blood pressure.

FIG. 8A presents the overall arrangement of the pressure applicator 2000, its holding strap 2400, the waveguide 3400, one end of which is connected to the pressure applicator via aperture 3450, the processing unit 3000 with microphone 3300 and pressure sensor 3200 connected to the other end of the waveguide 3400.

The pressure applicator contains several (two shown) pressure gauges 2700, functioning as a visual reminder to the user of the pressure that is to be achieved or maintained by the action of the pressure gauge visual indicator 2710 that in one of the embodiments of the disclosed invention is a piston that surfaces when applied pressure reaches a specific value, with the two depicted indicators becoming visible at 60 mmHg and 180 mmHg.

The first surface of the pressure applicator that for the purposes of measurement is applied against the skin above the arterial segment of choice, contains pressure equalizing elastic material 2050 and two rigid delineators 2500 that serve to compress the ends of the chosen arterial segment, thus assuring the segment is of specific predetermined length.

The second surface of the pressure applicator is equipped with a rigid concave support 2120 for the user's finger being at the same time the part of the pressure applicator to which the user applies applanation pressure during the measurement. The plane of the cross-section 3-3 is indicated with arrows.

FIG. 8B presents the schematic inset to illustrate arrangement and connections of the internal components E-E with the detailed depiction of the simplified pressure applicator 2000, which is comprised of the two surfaces and an internal cavity 2150. The pressure applicator is held in place by the holding strap 2400.

The first surface 2010 having the rigid delineators 2500 and pressure equalizing elastic material 2050 functioning to deliver equal pressure to a specific length of the arterial segment (not shown) as the user applies pressure to the second surface.

The second surface 2100 has the rigid concave support 2120 for the user's finger, which also serves as a source of sound when tapped/stricken, with said sound having defined frequencies and amplitudes and used for calibration of the microphone 3300 located in the processing unit 3000. The internal cavity 2150 an aperture 3450 hermetically connected to the waveguide 3400. The other end of the waveguide 3400 is hermetically connected to the processing unit pressure sensor 3200 and the processing unit microphone 3300.

As sound waves and variations of pressure are generated by the action of the user who applies pressure to the second surface 2100 of the pressure applicator causing the compression of the arterial segment (not shown) underneath, the internal cavity serves as a resonator for the sound waves and as an expansion/compression chamber, thus matching the pressure applied onto the arterial segment. Both the sound and the pressure are transmitted by the waveguide to the sensors of the processing unit that quantifies, records, and processes these signals.

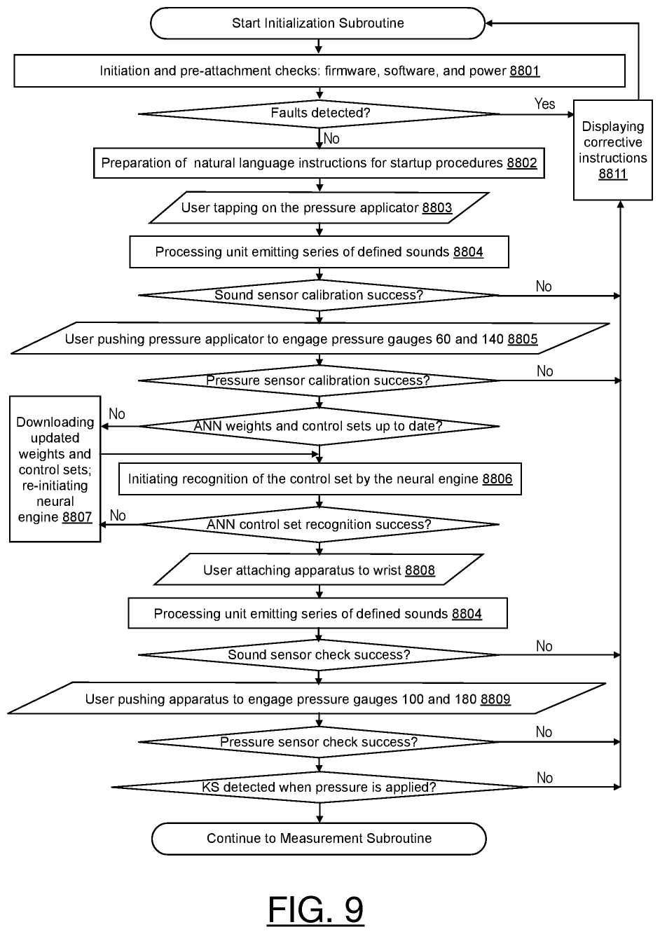

FIG. 9 depicts the outline of the initialization subroutine, comprised of the steps implemented by the user and operations performed by the processing unit.

The first step, upon powering on, with the components of the apparatus laid out within easy reach of the user, preferably on a flat surface, is comprised of firmware, software, and power checkups 8801 upon which, in case of fault detection, the appropriate chapter of the pre-compiled set of corrective instructions 8811 is supplied to the user via sound, voice, and graphical interfaces. These may include instructions to charge the power source or clean the apertures of sensors. Provisioning of corrective instructions causes the initiation subroutine to be re-initialized.

If no fault is detected, the processing unit prepares natural, easy-to-understand instructions for startup procedures 8802 and presents them to the user. The user performs the first step of sound sensor calibration by tapping 8803 on the pressure applicator, which generates characteristic vibrations and sounds. Since the pressure applicator is of defined dimensions, mass and composition, the tapping sound can be used for initial calibration of the sound processing systems of the disclosed apparatus.

The second step of calibration of the sound processing systems is the emission 8804 by the processing unit of a series of sounds defined by amplitude and frequency that are captured by the sound sensor and processed by the sound processing setups of the processing unit.

Sound sensor calibration may be unsuccessful, in which case the appropriate chapter of the pre-compiled set of corrective instructions 8811 is supplied to the user. These instructions may include instructions to shift the positioning of the pressure applicator, bring it closer to the processing unit, or clean the apertures of sensors.

In case of successful calibration of the sound processing systems, the following step 8805 is to instruct the user to apply progressively increasing amount of pressure onto the pressure applicator, pushing it against a hard, flat surface until the pressure gauges 60 and 140 become engaged.

Engagement may be demonstrated by a change in the appearance of the gauge: it may pop out of the body of the pressure applicator, it may change color or shape; alternatively the processing unit interface may indicate the engagement using voice, sound or graphical interface.

The results of pressure sensor calibration are interpreted by the processing unit for the correspondence of the engagement of the calibrated pressure gauges with the anticipated values of pressure magnitude and, in case of fault the appropriate chapter of the pre-compiled set of corrective instructions 8811 is supplied.

These may include instructions to clean the pressure applicator, to reposition it on a non-yielding surface, or to change the direction in which the pressure is applied to be truly perpendicular to the second surface of the pressure applicator.

In case of successful calibration of the pressure processing systems, the processing unit performs the self-check of the neural engine by affirming the presence of the weights of artificial neural networks formed in the neural engine and control sets for these artificial neural networks and putting the control sets through the neural engine so that control sets can be segmented and classified 8806.

In case of a fault, appropriate updated weights and control sets are obtained and the neural engine is reinitiated 8807 with the newly obtained weights. These may be obtained by additional training of the artificial neural network on the device itself or a pre-formed set may be downloaded from a server.

In case of the successful initiation of the neural engine, the processing unit instructs the user on the procedure of attachment 8808 of the pressure applicator to the selected area of the body, typically the wrist and proceeds with the sound sensor check by emitting a series of defined sounds 8804. In case the sound sensor check is unsuccessful, the appropriate chapter of the pre-compiled set of corrective instructions 8811 is supplied. These may include instructions to reposition the pressure applicator.

If the sound sensor check is successful, the user is instructed 8809 to apply pressure to the pressure applicator to engage pressure gauges 100 and 180. Engagement may be demonstrated by a change in the appearance of the gauge: it may pop out of the body of the pressure applicator, it may change color or shape; alternatively the processing unit interface may indicate the engagement using voice, sound or graphical interface.

The results of pressure sensor check are interpreted by the processing unit for the correspondence of the engagement of the calibrated pressure gauges with the anticipated values of pressure magnitude and, in case of fault the appropriate chapter of the pre-compiled set of corrective instructions 8811 is supplied. These may include instructions to reposition the pressure applicator, or to change the direction in which the pressure is applied to be truly perpendicular to the second surface of the pressure applicator.

During the pressure sensor check the processing unit also performs the functional check of detection of the Korotkoff sounds that arise in the compressed arterial segment with each pulse wave passage. Failure to generate Korotkoff sounds results in the processing unit supplying corrective instructions 8011 to the user and re-initiation of the initialization subroutine.

Successful completion of all of the listed steps allows the continuation to the measurement subroutine.

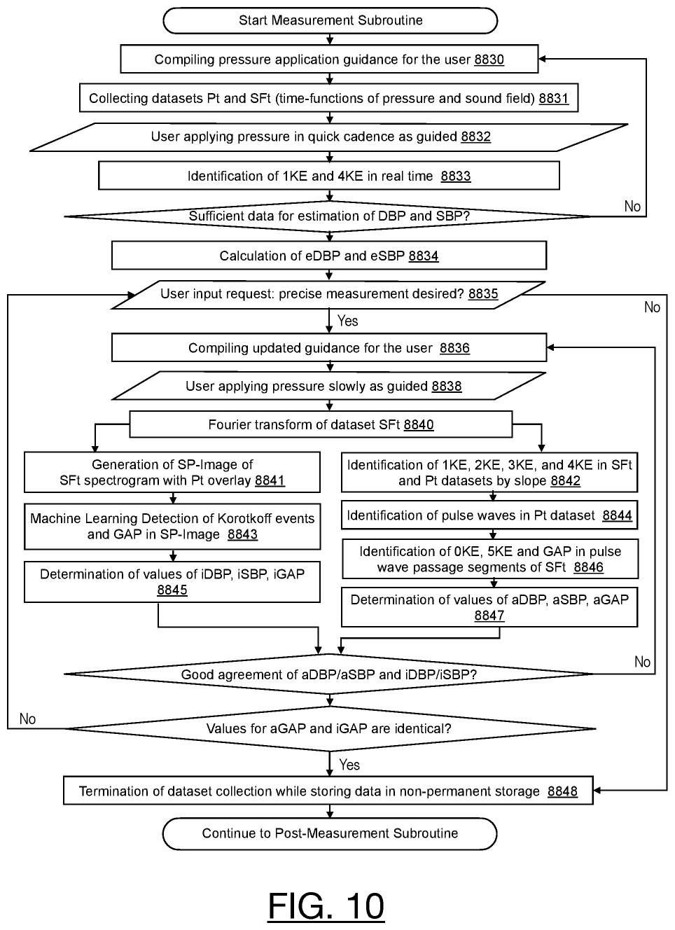

FIG. 10 depicts the outline of the initialization subroutine, comprised of the steps implemented by the user and operations performed by the processing unit.

The first step is the compilation of the initial guidance for the user 8830. This guidance, a pressure application profile, is comprised of easy-to-understand instructions to apply pressure in a quick cadence, to increase pressure, to hold pressure and to release pressure, each timed to specific duration, preferably not exceeding the duration of 2-4 pulse wave passages, and combined in a sequence that is communicated to the user via the processing unit interfaces as sound, voice, text, or image.

As the initial guidance is compiled, the processing unit begins to collect 8831 the datasets: Pt (pressure as a function of time) and SFt (sound field as a function of time), these are committed to volatile memory.

The user applies pressure 8832 onto the pressure applicator in a quick cadence as guided by the processing unit interface, while the processing unit identifies the 1st and the 4th Korotkoff events in real time. Notably, the 1st KE is the first appearance of sound as the fully compressed arterial segment becomes minimally passable for the pulse wave, and the 4th KE is the last sound detected as the arterial segment is no longer compressed.

Both of these events are edge events, with sound appearing after a period of silence (1KE) and disappearing followed by silence (4KE) and can be identified in real time 8833 without the use of neural network processing by a simple algorithm. Ordinarily, more than two 1KE and two 4KE need to be detected for the reliable estimation of diastolic blood pressure and systolic blood pressure; the initial assessment is repeated if insufficient number of 1KE and 4KE is detected with new adjusted guidance 8830 compiled for the user until the collected data is sufficient for calculation of the estimated diastolic blood pressure eDBP and estimated systolic blood pressure eSBP 8834.

The following step is the request 8835 of user's input whether a more precise measurement is desired. A negative answer causes the termination of dataset collection and commitment of datasets to the non-volatile memory, optionally also to an off-site storage 8848; while a positive answer causes the compilation 8836 of another, updated pressure application guidance.

As with the first guidance, this guidance is comprised of easy-to-understand instructions to increase and decrease the applied pressure communicated to the user via the processing unit interfaces as sound, voice, text, or image.

In contrast to the guidance utilized for the initial estimation, guidance for the precise measurement instructs the user to apply and release the pressure more slowly, taking approximately 4-5 pulse waves both to reach the peak pressure and to reduce the pressure to zero. The user is instructed to hold for 1-2 pulse waves once maximum pressure has been reached, wherein the value for maximum pressure is determined as 140% of eSBP as obtained at step 8834.

As the user applies the pressure slowly as guided 8838, the SFt dataset is subjected to Fourier transform 8840 of time-function data into frequency-function data allowing for determination of characteristic frequencies. Transformed dataset FF, original datasets SFt and Pt are further processed in parallel and in near-real time.

A SP-Image, a bitmap graphical image, is generated 8841 by representing dataset FF as a spectrogram, wherein the horizontal coordinate is time, the vertical coordinate is frequency and the amplitude of each characteristic frequency is represented by grayscale density.

The spectrogram is overlayed with a representation of the magnitude of the applied pressure as a curve with the horizontal coordinate is time, the vertical coordinate is pressure magnitude and the curve is comprised of connected datapoints of the dataset Pt assigned pseudocolor, preferably indexed to be distinct from the grayscale spectrogram.

Importantly, pressure fluctuations associated with the passage of pulse waves are not subject to smoothing or averaging to provide information necessary for detection of the ‘silent’ Korotkoff events, 0KE and 5KE.

The machine learning setup, realized as an artificial neural network subjects the graphical image to analysis, detecting 8843 all of the Korotkoff events and determining the presence or absence of the clinical phenomenon known as the auscultatory gap.

Values denoted as iSBP, iDBP, and iGAP are assigned based on the KE and GAP detection 8845 in the graphical image. Of note is the fact that advances in machine learning are especially notable in analysis of graphical images—from optical character recognition to ‘creation’ of artwork, making the task of recognition of KE and GAP achievable with a reasonably small neural engine and in near-real time.

In parallel with steps 8841, 8843, and 8845, an algorithmic analysis of datasets is performed to generate an independent set of values for blood pressure parameters and presence of the auscultatory gap.

The algorithmic analysis is comprised of the three consecutive steps.

Step 8842 is the identification of 1 KE, 2KE, 3KE, and 4KE in SFt and FF datasets on the basis of characteristic frequencies and amplitudes.

Step 8844 is the identification of pulse waves in the Pt dataset as fluctuations of pressure caused by the pulse wave encountering the impediment in the form of the pressure applicator.

The final step, 8846 is the identification of 0KE and 5KE, the silent Korotkoff events.

0KE is identified as corresponding to at least one pulse wave that does not generate sound when applied pressure is higher than the estimated SBP (eSBP from step 8834) and immediately precedes 1KE.

5KE is identified as corresponding to at least one pulse wave that does not generate sound when applied pressure is lower than the estimated DBP (eDBP from step 8834) and immediately follows 5KE.

The presence or absence of GAP is determined by the presence or absence of a drop in sound intensity exceeding 50 percent of the intensity of the sounds caused by the preceding and by the following pulse wave passage, providing the drop occurs during 2KE or 3KE but not any other Korotkoff event.

The final step in the algorithmic analysis is 8847, determination of values denoted as aSBP, aDBP, and aGAP.

The final steps of analysis are determined by the degree of agreement between aSBP and aDBP, as well as between iSPB and iSBP. In case of a poor agreement, the subroutine requests user's input 8835 whether the precise measurement is still desired.

A positive answer causes steps from 8836 to 8847 to be repeated with updated user guidance. A negative answer causes termination of the collection of datasets and places data in the non-permanent storage 8848.

In the case of the good agreement between aSBP and aDBP, as well as between iSPB and iSBP, with individual variance not exceeding a predetermined value including values set by medical instrument certification authorities, the degree of agreement between aGAP and iGAP is assessed and the absence of agreement returns the subroutine to step 8835, requesting user's input whether the precise measurement is still desired.

A positive answer causes steps from 8836 to 8847 to be repeated with updated user guidance. A negative answer causes termination of the collection of datasets and places data in the non-permanent storage 8848.

In the case of between aSBP and aDBP, as well as between iSPB and iSBP, as well as a positive agreement of aGAP and iGAP, the final step of the measurement subroutine 8848 is performed, wherein dataset collection is stopped and data are committed to the non-permanent storage (RAM) of the processing unit. Performance of step 8848 signifies the end of the measurement subroutine and the processing unit continues with the post-measurement subroutine.

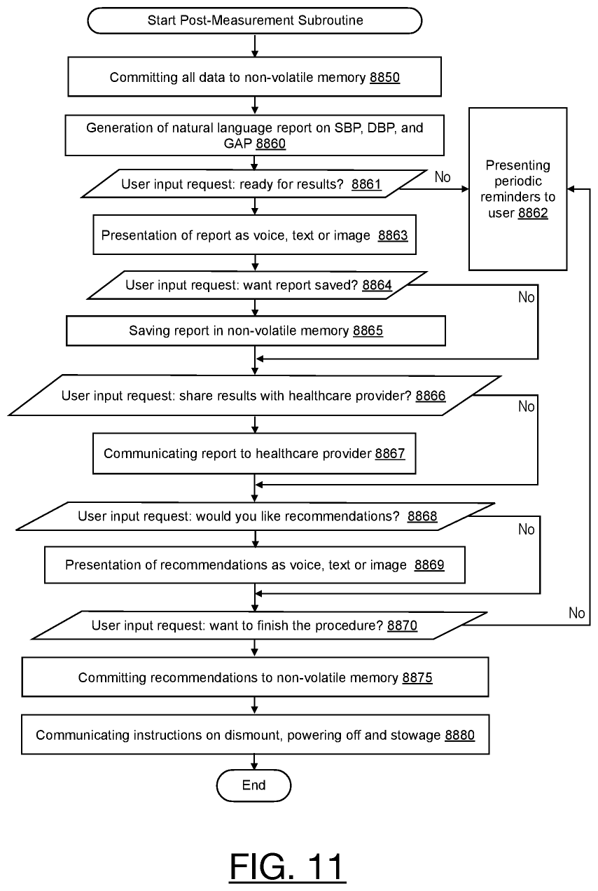

FIG. 11 depicts the steps of the post-measurement subroutine implemented by the processing unit with multiple user inputs.

The first step of the post-measurement subroutine is committing 8850 all collected data to non-volatile memory as a backup.

Following the backup of data, the natural language setup of the processing unit generates an easy-to-understand and personalized report 8860 on the values of SBP, DBP, and GAP with the reported value for DBP being the average of aDBP and iDBP, and the reported value for SBP being the average of aSBP and iSBP, with the value of GAP being reported as “present” or “absent” as the logical disjunction of the values of iGAP and aGAP.

The user is requested 8861 input on being ready to receive the report. A negative answer places the subroutine on hold with periodic reminders to the user 8862 that a report is ready to be viewed.

A positive answer causes the processing unit to present 8863 the report as voice, text, or image as per predetermined user preferences and request user's input 8864 on whether the report should be saved. An affirmative input causes the processing unit to save 8865 the report in non-volatile memory and proceed to step 8866, while a negatory input causes the processing unit to proceed to step 8866 directly.

User input is requested 8866 whether to share the results with a healthcare provider. A negative answer causes the processing unit to proceed to step 8868, a positive answer causes the processing unit to proceed to step 8867 and communicate the results of the measurement to the user-specified healthcare provider, followed by step 8868.

Additional user input is requested 8868 for presentation of health-related recommendations based on the results of the measurement. A negative answer causes the processing unit to proceed to step 8870, while a positive answer causes the processing unit to present 8869 an easy-to-understand and personalized set of natural language recommendations including dietary, lifestyle, medication and procedure compliance as well as reminders of appointments and similar health-related information. The report is presented to the user as voice, text, or image as per predetermined user preferences.

The final steps of the post-measurement subroutine are the request for user input 8870 on whether the user wants to finish the procedure. In case of a negative answer the subroutine implements step 8862, presenting periodic reminders to user that the procedure has not been finalized. A positive answer causes the processing unit to commit 8875 recommendations to non-volatile memory and communicate 8880 instructions on dismounting the apparatus, powering it off and stowage.

### REFERENCE SIGNS LIST

1000—Pulse wave
1010—Korotkoff sounds amplitude as a function of time
1020—Korotkoff sounds spectrogram
1050—User-applied force
1070—Pressure recorded as a function of time
1080—Diastolic arterial blood pressure (DBP)
1090—Systolic arterial blood pressure (SBP)
1100—Korotkoff events
1101—Korotkoff event 1
1102—Korotkoff event 2
1103—Korotkoff event 3
1104—Korotkoff event 4
1105—Korotkoff event 5
1110—Korotkoff event 0
1120—Sound amplitude as a function of time curve
1121—Tangent line (derivative) at a given point of a curve
1122—Slope in radians of the tangent line at a given point of a curve
1200—Auscultatory gap
1400—Arterial segment
1410—Compressed arterial segment
1500—Soft tissue
1510—Radius (bone)
1511—Ulna (bone)
1600—User's finger
2000—Pressure applicator
2010—First surface of the pressure applicator
2050—Pressure equalizing elastic material
2100—Second surface
2120—Rigid concave support
2150—Internal cavity
2200—Pressure applicator pressure sensor
2300—Pressure applicator microphone
2400—Holding strap (bracelet)
2500—Rigid delineator
2700—Pressure gauge setup
2710—Pressure gauge visual indicator
2720—Pressure gauge elastic element
2730—Pressure gauge mounting
2800—Pressure applicator's wireless antenna
2820—Communication (signal) bus
2850—Pressure applicator induction coil
2890—Pressure applicator power source
3000—Processing Unit
3200—Processing unit pressure sensor
3300—Processing unit microphone
3400—Waveguide
3450—Waveguide aperture inside the pressure applicator
3700—CPU (Central Processing Unit)
3701—Communication Controller
3702—Power Controller
3703—Sound Pre-Processing Setup
3704—Pressure Pre-Processing Setup
3705—Fourier Transform Setup
3706—Neural Engine (Machine Learning Setup)
3707—Natural Language Processing
3708—Interface Controller
3711—Non-permanent storage, RAM (Random Access Memory)
3712—Non-volatile storage
3709—Acoustic Interface
3710—Visual Interface
3720—Additional Processing and Storage (Cloud, Off-site)
3800—Processing unit wireless antenna
3850—Processing unit induction coil
3890—Processing unit power source
8801—Initiation and pre-attachment checks of firmware, software, and power
8802—Preparation of natural language instruction for startup procedures
8803—User tapping on the pressure applicator
8804—Processing unit emitting series of defined sounds
8805—User pushing pressure applicator to engage pressure gauges 60 mmHg and 140 mmHg
8806—Initiating recognition of the control set by the neural engine
8807—Downloading updated weights and control sets and re-initiating neural engine
8808—User attaching apparatus to wrist
8809—User pushing apparatus to engage pressure gauges 100 mmHg and 180 mmHg
8811—Displaying corrective instructions
8830—Compiling initial guidance for the user
8831—Collecting datasets Pt (time-function of pressure) and SFt (time-function of the
sound field)
8832—User applying pressure in quick cadence as guided
8833—Identification of 1KE and 4KE in real time
8834—Calculation of eDBP and eSBP
8835—User Input Request: Precise Measurement Desired?
8836—Compiling Updated Guidance for the User
8838—User applying pressure slowly as guided
8840—Fourier transform of dataset SFt
8841—Generation of SP-image from SFt spectrogram with Pt overlay
8842—Identification of 1KE, 2KE, 3KE, and 4KE in SFt and Pt datasets by slope
8843—Machine learning detection of Korotkoff events and GAP in SP-image
8844—Identification of pulse waves in Pt dataset
8845—Determination of values of iDBP, iSBP, iGAP
8846—Identification of 0KE, 5KE and GAP in pulse wave passage segments of SFt
8847—Determination of values of pDBP, pSBP, pGAP
8848—Termination of dataset collection while storing data in non-permanent storage
8850—Committing all data and reports to non-volatile memory
8860—Generation of natural language report on SBP, DBP, and GAP
8861—User input request: ready for results?
8862—Presenting periodic reminders to user
8863—Presentation of report as voice, text or image
8864—User input request: want report saved?
8865—Saving report and datasets in non-volatile memory
8866—User input request: share results with healthcare provider?
8867—Communicating report to healthcare provider
8868—User input request: would you like recommendations?
8869—Presentation of recommendations as voice, text or image
8870—User input request: want to finish the procedure?
8875—Committing recommendations to non-volatile memory
8880—Communicating instructions on dismount, powering off, and stowage

We claim:

1. An apparatus for measurement of arterial blood pressure comprising a pressure applicator with two working surfaces, and a processing unit connected to the pressure applicator by data interchange connection.

2. The apparatus of claim 1 further comprising a pressure sensor configured to measure pressure between 1 and 300 millimeters mercury with sampling frequency of no less than 50 Hz;
further comprising a sound sensor, configured to transduce a sound field characterized by frequencies between 50 and 1000 Hz and intensity between 10⁻¹¹ W/m2 and 10⁻⁶ W/m2;
further comprising a signal communication setup capable of establishing and
maintaining at least one-way wireless communication with nearby wireless receivers; and further comprising at least one source of electrical power selected from a group consisting of disposable electric batteries, rechargeable electric batteries, electric condensers, induction coils, and antennas configured to receive power-over-wireless.

3. The pressure applicator of claim 1 wherein the first working surface is applied against the soft tissue above an artery of the user and is configured with at least two rigid delineator elements separated by a fixed distance and a pressure equalizing elastic material element positioned between the rigid delineator elements; wherein the second working surface is configured with a rigid concave support shaped to provide comfortable support to the tip of a human finger and at least one pressure gauge configured to change appearance as the applied pressure becomes equal to a pre-established threshold.

4. The pressure applicator of claim 1 wherein the applanation pressure is provided by the action of the user's fingers applied to the rigid concave support of the second surface of the pressure applicator and causes compression of the arterial segment underneath the rigid delineators of the first working surface, with said compressed arterial segment generating sounds known as Korotkoff sounds of the turbulent flow of blood corresponding to the passage of a pulse wave through the compressed arterial segment.

5. The pressure applicator of claim 1 wherein the artery of the user is selected from the group consisting of radial artery of the upper extremity, ulnar artery of the upper extremity, brachial artery of the upper extremity, femoral artery of the lower extremity, popliteal artery of the lower extremity, tibialis posterior artery of the lower extremity, dorsalis pedis artery of the lower extremity, external carotid artery, facial artery, and temporal artery.

6. The processing unit of claim 1 further comprising: a power controller in operational connection with the source of electrical power; a non-permanent random access memory data storage setup, a long-term non-volatile data storage setup, and a system on a chip electronic assembly with dedicated circuitry for performance of binary computing operations selected from the group consisting of:
processing of readouts of sound sensors and pressure sensors with interconversion of analog and digital data;
Fourier transform of oscillatory signals into constituent frequencies with corresponding amplitudes;
assembly of datasets comprised of pressure magnitude as a function of time, sound amplitude as a function of time and sound amplitude as a function of frequency;
generation of spectrogram of sound amplitude and constituent frequencies as a graphical image that is a graphical map of sound frequencies and amplitudes with time as a parameter;
algorithmic recognition of changes in data values that exceed predetermined thresholds;
machine learning-based recognition of patterns as present in the dataset comprised of the first derivatives of the pressure magnitude as a function of time and sound amplitude as a function of time taken at regular intervals corresponding to passage of pulse waves;
machine learning-based recognition of patterns corresponding to Korotkoff events and auscultatory gap in the graphical image comprised of the spectrogram that is a graphical map of sound frequencies and amplitudes with time as a parameter overlayed with the curve representing pressure as a function of time;
updating of neural network weights and transfer functions for the purpose of sustaining and improving performance of machine learning computing operations;
generation of natural language instructions for the user containing directions regarding timing of application of applanation pressure, duration of application of applanation pressure, rate of change of the applied pressure, and direction of the vector of applied pressure;
generation of natural language reports on findings of arterial blood pressure, trends in arterial blood pressure as observed over time, presence or absence of the clinical phenomenon known as the auscultatory gap;
generation of natural language health-related recommendations for the user;
presentation of said instructions, reports, and recommendations to the user and third parties utilizing voice, sound, text, and graphical interactive interfaces;
encoding of datasets, instructions, and reports in conformance with applicable privacy laws and regulations;
storing the encoded datasets, instructions, and reports in long-term non-volatile data storage and offsite cloud storage.

7. A method for measurement of arterial blood pressure and detection of the clinical phenomenon known as the auscultatory gap wherein the improvement comprises:
the use of applanation pressure provided by the action of the user's fingers to compress the arterial segment in which blood pressure is measured and the presence of the auscultatory gap is detected to generate biophysical phenomena known as Korotkoff events (KE);
compression of one pre-selected arterial segment without the interruption of blood flow through collateral blood vessels, avoiding unpleasant sensations and venous congestion distally of the preselected arterial segment;
active participation of the user in the process of measurement, increasing the subjective feeling of being in control and reducing the risk of anxiety and associated transient arterial hypertension known as “white coat hypertension”;
provisioning and modification in real-time instructions to the user how and when to apply applanation pressure, change its magnitude and the direction of the vector of the applanation pressure with said instructions selected from a group consisting of voice-based, sound-based, text-based, and image-based directives;
utilization of the more natural and easy to produce pulsatile application of applanation pressure in place of a steady and slow increase and decrease of applanation force that is overly demanding of the user;
collecting at least two datasets from the target arterial segment selected from the group consisting of pressure magnitude as a function of time dataset obtained with a pressure sensor, sound field intensity as a function of time dataset obtained with a sound sensor, and sound amplitude as a function of sound frequency dataset;
generation of the sound spectrogram image in real time as a constantly updating and elongating with the passage of time two-dimensional graphical image combining the sound amplitude as a function of time dataset with the sound amplitude as a function of frequency dataset and overlayed with a curve representing pressure magnitude as a function of time dataset;
identifying the passage of each pulse wave through the arterial segment by monitoring the elevation of the pressure magnitude within the arterial segment;
identifying 1st and 4th Korotkoff events in real time as watershed events occurring during passage of pulse waves through the compressed arterial segment characterized by the abrupt change in sound amplitude as compared to the background noise;
estimating of the systolic blood pressure as corresponding to 1st Korotkoff events, and the diastolic blood pressure as corresponding to 4th Korotkoff events;
adjusting user instructions to reflect the estimated systolic and diastolic blood pressure in regard to magnitude of the applanation pressure, duration of its application, timing of its application, and the direction of the vector of the applied pressure;
obtaining the more precise values of systolic and diastolic blood pressure by comparing systolic and diastolic blood pressure values obtained utilizing two distinct and complementary modalities: the first modality being machine learning-based recognition of 1st, 2nd, 3rd, and 4th Korotkoff events in a two-dimensional graphical image comprised of the sound spectrogram with a pressure data overlay, and the second modality being machine learning-based recognition of all Korotkoff events, including 0th and 5th in the combined datasets based on establishment of correspondence between first derivatives of pressure magnitude as a function of time and sound amplitude as a function of time as values of said derivatives change with passage of pulse waves;
performing self-validation of the more precise measurement by establishing correspondence between values obtained by the two distinct machine learning-based modalities with said correspondence being no less than a predetermined value, typically 85 percent or more;
determining the presence or absence of the clinical phenomenon known as the auscultatory gap utilizing two distinct modalities: the first modality being machine learning-based recognition of a drop of sound intensity during 2nd or 3rd Korotkoff events in a two-dimensional graphical image comprised of the sound spectrogram with pressure data overlay, and the second modality being machine learning-based recognition of the pattern in the values of first derivatives of pressure magnitude as a function of time and sound amplitude obtained during 2nd and 3rd Korotkoff events;
determining the need for repeated and adjusted measurement based on a predetermined degree but no less than 85% correspondence between the value of systolic and diastolic blood pressure obtained by the two distinct methods and the presence or absence of the clinical phenomenon known as the auscultatory gap as established by the two distinct methods;
provisioning of a natural language easy-to-understand report to the user with blood pressure values and indication of the presence of the clinical phenomenon known as the auscultatory gap;
optional provisioning of a report containing blood pressure values and indication of the presence of the clinical phenomenon known as the auscultatory gap to a pre-designated health care provider;
optional provisioning of a natural language easy-to-understand lifestyle and health-related measures recommendations to the user.

8. The method of claim 7 wherein:
the 1st Korotkoff event is defined as the first appearance of louder than background sound as the externally applied applanation pressure approximates the systolic pressure to such extent that the previously fully compressed arterial segment becomes minimally permissive for the passage of a pulse wave;
the 2nd Korotkoff event is defined as the increase of loudness of the sound generated by the passage of pulse waves as the degree of obturation of the arterial segment by the externally applied applanation pressure diminishes from nearly 1.0 to approximately 0.66-0.5 and string-like vibrations predominate generating frequencies in the 50 to 200 Hz range;
the 3rd Korotkoff event is defined as the further increase of loudness of the sound generated by the passage of pulse waves as the degree of obturation of the arterial segment by the externally applied applanation pressure diminishes from approximately 0.66-0.5 and cylinder/pipe-like vibrations predominate causing enrichment of the sound with frequencies in the 200 to 350 Hz range;
the 4th Korotkoff event is defined as the last appearance of louder than background sound as the externally applied applanation pressure approximates the diastolic pressure to such extent that the compressed arterial segment is minimally impeding the passage of the pulse wave;
the 0th Korotkoff event is defined as the absence of louder than background sound during the pulse wave that fails to pass through the compressed arterial segment as the externally applied applanation pressure exceeds the systolic arterial blood pressure;
the 5th Korotkoff event is defined as the absence of louder than background sound during the pulse wave as the arterial segment is not substantially compressed or deformed and the externally applied applanation pressure does not exceed the diastolic arterial blood pressure;
the clinical phenomenon known as the auscultatory gap is defined as at least a 40 percent drop in amplitude of pulse wave generated sound during the 2nd Korotkoff event or 3rd Korotkoff event that lasts at least the duration of one pulse wave.
